SEQUENCE LISTING (4 sequences)

SEQ ID NO: 1 - DNA, 12641 bp, Artificial
  vector
agcttgcgatcgctagggataacagggtaatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcggatcctagacgcgtgagatcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggtccatggttttggtttaataagaagagaaaagagttcttttgttatggctgaagtaatagagaaatgagctcgagtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttgacgaagattttcttcttgtcattgagtcgtaaaagactctgtatgaactgttcgccagtcttcacggcgagttctgttagatcctcgatctgaatttttgactccatgtatggtgcatatggcgcgcctaagctagctagatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagatacgtattaccctgttatccctaaagcttatcgatttcgaacccctcaggcgaagaacaggtatgatttgtttgtaattagatcaggggtttaggtctttccattactttttaatgttttttctgttactgtctccgcgatctgattttacgacaatagagtttcgggttttgtcccattccagtttgaaaataaaggtccgtcttttaagtttgctggatcgataaacctgtgaagattgagtctagtcgatttattggatgatccattcttcatcgtttttttcttgcttcgaagttctgtataaccagatttgtctgtgtgcgattgtcattacctagccgtgtatcgagaactagggttttcgagtcaattttgccccttttggttatatctggttcgataacgattcatctggattagggttttaagtggtgacgtttagtattccaatttcttcaaaatttagttatggataatgaaaatccccaattgactgttcaatttcttgttaaatgcgcagatccccatggcttcgatctcctcctcagtcgcgaccgttagccggaccgcccctgctcaggccaacatggtggctccgttcaccggccttaagtccaacgccgccttccccaccaccaagaaggctaacgacttctccacccttcccagcaacggtggaagagttcaatgtatgcaggtgtggccggcctacggcaacaagaagttcgagacgctgtcgtacctgccgccgctgtctatggcgcccaccgtgatgatggcctcgtcggccaccgccgtcgctccgttccaggggctcaagtccaccgccagcctccccgtcgcccgccgctcctccagaagcctcggcaacgtcagcaacggcggaaggatccggtgcatggccggcgccgaggagatcgtgctgcagcccatcaaggagatctccggcaccgtcaagctgccggggtccaagtcgctttccaaccggatcctcctactcgccgccctgtccgaggggacaacagtggttgataacctgctgaacagtgaggatgtccactacatgctcggggccttgaggactcttggtctctctgtcgaagcggacaaagctgccaaaagagctgtagttgttggctgtggtggaaagttcccagttgaggatgctaaagaggaagtgcagctcttcttggggaatgctggaatcgcaatgcggtccttgacagcagctgttactgctgctggtggaaatgcaacttacgtgcttgatggagtaccaagaatgagggagagacccattggcgacttggttgtcggattgaagcagcttggtgcagatgttgattgtttccttggcactgactgcccacctgttcgtgtcaatggaatcggagggctacctggtggcaaggtcaagctgtctggctccatcagcagtcagtacttgagtgccttgctgatggctgctcctttggctcttggggatgtggagattgaaatcattgataaattaatctccattccgtacgtcgaaatgacattgagattgatggagcgttttggtgtgaaagcagagcattctgatagctgggacagattctacattaagggaggtcaaaaatacaagtcccctaaaaatgcctatgttgaaggtgatgcctcaagcgcaagctatttcttggctggtgctgcaattactggagggactgtgactgtggaaggttgtggcaccaccagtttgcagggtgatgtgaagtttgctgaggtactggagatgatgggagcgaaggttacatggaccgagactagcgtaactgttactggcccaccgcgggagccatttgggaggaaacacctcaaggcgattgatgtcaacatgaacaagatgcctgatgtcgccatgactcttgctgtggttgccctctttgccgatggcccgacagccatcagagacgtggcttcctggagagtaaaggagaccgagaggatggttgcgatccggacggagctaaccaagctgggagcatctgttgaggaagggccggactactgcatcatcacgccgccggagaagctgaacgtgacggcgatcgacacgtacgacgaccacaggatggcgatggctttctcccttgccgcctgtgccgaggtccccgtcaccatccgggaccctgggtgcacccggaagaccttccccgactacttcgatgtgctgagcactttcgtcaagaattaagctctagaactagtggatcccccgatccgcgtttgtgttttctgggtttctcacttaagcgtctgcgttttacttttgtattgggtttggcgtttagtagtttgcggtagcgttcttgttatgtgtaattacgctttttcttcttgcttcagcagtttcggttgaaatataaatcgaatcaagtttcactttatcagcgttgttttaaattttggcattaaattggtgaaaattgcttcaattttgtatctaaatagaagagacaacatgaaattcgacttttgacctcaaatcttcgaacatttatttcctgatttcacgatggatgaggataacgaaagggcggttcctatgtccgggaaagttcccgtagaagacaatgagcaaagctactgaaacgcggacacgacgtcgcattggtacggatatgagttaaaccgactcaattcctttattaagacataaaccgattttggttaaagtgtaacagtgagctgatataaaaccgaaacaaaccggtacaagtttgattgagcaacttgatgacaaacttcagaattttggttattgaatgaaaatcatagtctaatcgtaaaaaatgtacagaagaaaagctagagcagaacaaagattctatattctggttccaatttatcatcgctttaacgtccctcagatttgatcgggctgcaggaattaaacgcccgggcacgtgggatcctctagagtcgacggccgagtactggcaggatatataccgttgtaatttgtcgcgtgtgaataagtcgctgtgtatgtttgtttgattgtttctgttggagtgcagcccatttcaccggacaagtcggctagattgatttagccctgatgaactgccgaggggaagccatcttgagcgcggaatgggaatggatttcgttgtacaacgagacgacagaacacccacgggaccgagcttcgatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgccgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatccgaatctggcaattccggttcgcttgctgtccataaaaccgcccagtctagctatcgccatgtaagcccactgcaagctacctgctttctctttgcgcttgcgttttccggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcgatccctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgcatgcccgttccatacagaagctgggcgaacaaacgatgctcgccttccagaaaaccgaggatgcgaaccacttcatccggggtcagcaccaccggcaagcgcccggacggccgaggtcttccgatctcctgaagccagggcagatccgtgcacagcacttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctagcttgcttggtcgttccggtaccgtgaacgtcggctcgattgtacctgcgttcaaatactttgcgatcgtgttgcgcgcctgcccggtgcgtcggctgatctcacggatcgactgcttctctcgcaacgccatccgacggatgatgtttaaaagtcccatgtggatcactccgttgccccgtcgctcaccgtgttggggggaaggtgcacatggctcagttctcaatggaaattatctgcctaaccggctcagttctgcgtagaaaccaacatgcaagctccaccgggtgcaaagcggcagcggcggcaggatatattcaattgtaaatggctccatggcgatcgctacgtatctagaattcctgcaggtcgagtcgcgacgtacgttcgaacaattggttttaaa

SEQ ID NO: 2 - DNA, 12188 bp, Artificial
  vector
cggcaggatatattcaattgtaaatggctccatggcgatcgctacgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggtccatggttttggtttaataagaagagaaaagagttcttttgttatggctgaagtaatagagaaatgagctcgagtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttgacgaagattttcttcttgtcattgagtcgtaaaagactctgtatgaactgttcgccagtcttcacggcgagttctgttagatcctcgatctgaatttttgactccatgtatggtgcatatggcgcgcctaagctagctagatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagatacgtatctagaattcgaaggtaattatccaagatgtagcatcaagaatccaatgtttacgggaaaaactatggaagtattatgtgagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtctcgagatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgctatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcgcctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggatatcaagcttggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatctaactataacggtcctaaggtagcgaccgcgggagccatttgggaggaaacacctcaaggcgattgatgtcaacatgaacaagatgcctgatgtcgccatgactcttgctgtggttgccctctttgccgatggcccgacagccatcagagacgtggcttcctggagagtaaaggagaccgagaggatggttgcgatccggacggagctaaccaagctgggagcatctgttgaggaagggccggactactgcatcatcacgccgccggagaagctgaacgtgacggcgatcgacacgtacgacgaccacaggatggcgatggctttctcccttgccgcctgtgccgaggtccccgtcaccatccgggaccctgggtgcacccggaagaccttccccgactacttcgatgtgctgagcactttcgtcaagaattaagctctagaactagtggatcccccgatccgcgtttgtgttttctgggtttctcacttaagcgtctgcgttttacttttgtattgggtttggcgtttagtagtttgcggtagcgttcttgttatgtgtaattacgctttttcttcttgcttcagcagtttcggttgaaatataaatcgaatcaagtttcactttatcagcgttgttttaaattttggcattaaattggtgaaaattgcttcaattttgtatctaaatagaagagacaacatgaaattcgacttttgacctcaaatcttcgaacatttatttcctgatttcacgatggatgaggataacgaaagggcggttcctatgtccgggaaagttcccgtagaagacaatgagcaaagctactgaaacgcggacacgacgtcgcattggtacggatatgagttaaaccgactcaattcctttattaagacataaaccgattttggttaaagtgtaacagtgagctgatataaaaccgaaacaaaccggtacaagtttgattgagcaacttgatgacaaacttcagaattttggttattgaatgaaaatcatagtctaatcgtaaaaaatgtacagaagaaaagctagagcagaacaaagattctatattctggttccaatttatcatcgctttaacgtccctcagatttgatcgggctgcaggaattaaacgcccgggcacgtgggatcctctagagtcgacggccgagtactggcaggatatataccgttgtaatttgtcgcgtgtgaataagtcgctgtgtatgtttgtttgattgtttctgttggagtgcagcccatttcaccggacaagtcggctagattgatttagccctgatgaactgccgaggggaagccatcttgagcgcggaatgggaatggatttcgttgtacaacgagacgacagaacacccacgggaccgagcttcgatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgccgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatccgaatctggcaattccggttcgcttgctgtccataaaaccgcccagtctagctatcgccatgtaagcccactgcaagctacctgctttctctttgcgcttgcgttttccggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcgatccctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgcatgcccgttccatacagaagctgggcgaacaaacgatgctcgccttccagaaaaccgaggatgcgaaccacttcatccggggtcagcaccaccggcaagcgcccggacggccgaggtcttccgatctcctgaagccagggcagatccgtgcacagcacttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctagcttgcttggtcgttccggtaccgtgaacgtcggctcgattgtacctgcgttcaaatactttgcgatcgtgttgcgcgcctgcccggtgcgtcggctgatctcacggatcgactgcttctctcgcaacgccatccgacggatgatgtttaaaagtcccatgtggatcactccgttgccccgtcgctcaccgtgttggggggaaggtgcacatggctcagttctcaatggaaattatctgcctaaccggctcagttctgcgtagaaaccaacatgcaagctccaccgggtgcaaagcggcagcgg

SEQ ID NO: 3 - DNA, 11500 bp, Artificial
  vector
cggcaggatatattcaattgtaaatggctccatggcgatcgctaccctctgcagtcgacgggcccgttgtcccgcggtcgctaccttaggaccgttatagttagatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaaatagagagagatagatttgtagagagagactggtgatttcagcgtgtccaagcttgatatcctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggaggcgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatagcatccatggcctccgcgaccggctgcagaacagcgggcagttcggtttcaggcaggtcttgcaacgtgacaccctgtgcacggcgggagatgcaataggtcaggctctcgctgaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatctcgagacaaacttacaaatttctctgaagttgtatcctcagtacttcaaagaaaatagcttacaccaaattttttcttgttttcacaaatgccgaacttggttccttatataggaaaactcaagggcaaaaatgacacggaaaaatataaaaggataagtagtgggggataagattcctttgtgataaggttactttccgcccttacattttccaccttacatgtgtcctctatgtctctttcacaatcaccgaccttatcttcttcttttcattgttgtcgtcagtgcttacgtcttcaagattcttttcttcgcctggttcttctttttcaatttctacgtattcttcttcgtattctggcagtataggatcttgtatctgtacattcttcatttttgaacataggttgcatatgtgccgcatattgatctgcttcttgctgagctcacataatacttccatagtttttcccgtaaacattggattcttgatgctacatcttggataattaccttcgaattcgtcttggggatccgatatctagagtatacgcgttaacgcggccgctgtaccatgcatgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaaatagagagagatagatttgtagagagagactggtgatttcagcgtgtccaagcttgctagccctatttcaggaaagtttcggaggagatagtgttcggcagtttgtacatcatctgcgggatcaggtacggtttgatcaggttgtagaagatcaggtaagacatagaatcgatgtagatgatcggtttgtttttgttgatttttacgtaacagttcagttggaatttgttacgcagacccttaaccaggtattctacttcttcgaaagtgaaagactgggtgttcagtacgatcgatttgttggtagagtttttgttgtaatcccatttaccaccatcatccatgaaccagtatgccagagacatcggggtcaggtagttttcaaccaggttgttcgggatggtttttttgttgttaacgatgaacaggttagccagtttgttgaaagcttggtgtttgaaagtctgggcgccccaggtgattaccaggttacccaggtggttaacacgttcttttttgtgcggcggggacagtacccactgatcgtacagcagacatacgtggtccatgtatgctttgtttttccactcgaactgcatacagtaggttttaccttcatcacgagaacggatgtaagcatcacccaggatcagaccgatacctgcttcgaactgttcgatgttcagttcgatcagctgggatttgtattctttcagcagtttagagttcggacccaggttcattacctggttttttttgatgttaaccttgcgcttcttcttggggggtttagccatggttttggacttcttcttcttcttcttttgcttaattctcgagtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttgacgaagattttcttcttgtcattgagtcgtaaaagactctgtatgaactgttcgccagtcttcacggcgagttctgttagatcctcgatctgaatttttgactccatgtatggtgcatatggcgcgccatgcatacgtaggtaccaattgcctgcaggtcgacggccgagtactggcaggatatataccgttgtaatttgtcgcgtgtgaataagtcgctgtgtatgtttgtttgattgtttctgttggagtgcagcccatttcaccggacaagtcggctagattgatttagccctgatgaactgccgaggggaagccatcttgagcgcggaatgggaatggatttcgttgtacaacgagacgacagaacacccacgggaccgagcttcgatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgccgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatccgaatctggcaattccggttcgcttgctgtccataaaaccgcccagtctagctatcgccatgtaagcccactgcaagctacctgctttctctttgcgcttgcgttttccggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcgatccctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgcatgcccgttccatacagaagctgggcgaacaaacgatgctcgccttccagaaaaccgaggatgcgaaccacttcatccggggtcagcaccaccggcaagcgcccggacggccgaggtcttccgatctcctgaagccagggcagatccgtgcacagcacttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctagcttgcttggtcgttccggtaccgtgaacgtcggctcgattgtacctgcgttcaaatactttgcgatcgtgttgcgcgcctgcccggtgcgtcggctgatctcacggatcgactgcttctctcgcaacgccatccgacggatgatgtttaaaagtcccatgtggatcactccgttgccccgtcgctcaccgtgttggggggaaggtgcacatggctcagttctcaatggaaattatctgcctaaccggctcagttctgcgtagaaaccaacatgcaagctccaccgggtgcaaagcggcagcgg

SEQ ID NO: 4 - DNA, 10443 bp, Artificial
  vector
agattcgaagctcggtcccgtgggtgttctgtcgtctcgttgtacaacgaaatccattcccattccgcgctcaagatggcttcccctcggcagttcatcagggctaaatcaatctagccgacttgtccggtgaaatgggctgcactccaacagaaacaatcaaacaaacatacacagcgacttattcacacgcgacaaattacaacggtatatatcctgccagtactcggccgtcgacctgcaggcaattggtacctacgtatgcatggcgcgccatatgcaccatacatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactcgagaattaagcaaaagaagaagaagaagaagtccaaaaccatggctaaaccccccaagaagaagcgcaaggttaacatcaaaaaaaaccaggtaatgaacctgggtccgaactctaaactgctgaaagaatacaaatcccagctgatcgaactgaacatcgaacagttcgaagcaggtatcggtctgatcctgggtgatgcttacatccgttctcgtgatgaaggtaaaacctactgtatgcagttcgagtggaaaaacaaagcatacatggaccacgtatgtctgctgtacgatcagtgggtactgtccccgccgcacaaaaaagaacgtgttaaccacctgggtaacctggtaatcacctggggcgcccagactttcaaacaccaagctttcaacaaactggctaacctgttcatcgttaacaacaaaaaaaccatcccgaacaacctggttgaaaactacctgaccccgatgtctctggcatactggttcatggatgatggtggtaaatgggattacaacaaaaactctaccaacaaatcgatcgtactgaacacccagtctttcactttcgaagaagtagaatacctggttaagggtctgcgtaacaaattccaactgaactgttacgtaaaaatcaacaaaaacaaaccgatcatctacatcgattctatgtcttacctgatcttctacaacctgatcaaaccgtacctgatcccgcagatgatgtacaaactgccgaacactatctcctccgaaactttcctgaaatagggctagcaagcttggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcatgcatggtacagcggccgcgttaacgcgtatactctagagcgatcgccatggagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttaaacatcatccgtcggatggcgttgcgagagaagcagtcgatccgtgagatcagccgacgcaccgggcaggcgcgcaacacgatcgcaaagtatttgaacgcaggtacaatcgagccgacgttcacggtaccggaacgaccaagcaagctagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaagtgctgtgcacggatctgccctggcttcaggagatcggaagacctcggccgtccgggcgcttgccggtggtgctgaccccggatgaagtggttcgcatcctcggttttctggaaggcgagcatcgtttgttcgcccagcttctgtatggaacgggtcgacgcgtttaatgaccagcacagtcgtgatggcaaggtcagaatagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctattctgaccttgccatcacgactgtgctggtcattaaacgcgtcgacggatcagtgagggtttgcaactgcgggtcaaggatctggatttcgatcacggcacgatcatcgtgcgggagggcaagggctccaaggatcgggccttgatgttacccgagagcttggcacccagcctgcgcgagcagggatcgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatccggaaaacgcaagcgcaaagagaaagcaggtagcttgcagtgggcttacatggcgatagctagactgggcggttttatggacagcaagcgaaccggaattgcc

(19) United States
(12) Patent Application Publication
(10) Pub. No.: US 2014/0173770 A1
(43) Pub. Date: Jun. 19, 2014

(54) METHODS AND MEANS TO MODIFY A PLANT GENOME AT A PRESELECTED SITE

(71) Applicant: Bayer CropScience NV, Diegem (BE)

(72) Inventor: Katelijn D'Halluin, Mariakerke (BE)

(73) Assignee: Bayer CropScience NV, Diegem (BE)

(21) Appl. No.: 14/122,283

(22) PCT Filed: May 30, 2012
(86) PCT No.: PCT/EP2012/060181

§ 371 Date: Feb. 18, 2014

Related U.S. Application Data
(60) Provisional application No. 61/493,579, filed on Jun. 6, 2011.

(30) Foreign Application Priority Data
Jun. 6, 2011 (EP) 11004570.5

(51) Int. Cl. C12N 15/82; A01G 1/00

(52) U.S. Cl. CPC C12N 15/8205; C12N 15/8207; A01G 1/001
USPC 800/260; 435/468; 435/470; 435/469; 800/278; 435/410; 800/314; 435/419; 47/58.1 R

(57) ABSTRACT

Methods and means are provided to modify in a targeted manner the genome of a cotton plant using a double stranded DNA break inducing enzyme and embryogenic callus.

### FIELD OF THE INVENTION

The invention relates to the field of agronomy. More particularly, the invention provides methods and means to introduce a targeted modification, including insertion, deletion or substitution, at a precisely localized nucleotide sequence in the genome of a cotton plant using embryogenic callus.

### BACKGROUND ART

The need to introduce targeted modifications in plant genomes, including the control over the location of integration of foreign DNA in plants has become increasingly important, and several methods have been developed in an effort to meet this need (for a review see Kumar and Fladung, 2001, Trends in Plant Science, 6, pp 155-159). These methods mostly rely on the initial introduction of a double stranded DNA break at the targeted location via expression of a double strand break inducing (DSBI) enzyme.

Activation of the target locus and/or repair or donor DNA through the induction of double stranded DNA breaks (DSB) via rare-cutting endonucleases, such as I-SceI has been shown to increase the frequency of homologous recombination by several orders of magnitude. (Puchta et al., 1996, Proc. Natl. Acad. Sci. U.S.A., 93, pp 5055-5060; Chilton and Que, Plant Physiol., 2003; D'Halluin et al. 2008 Plant Biotechnol. J. 6, 93-102).

WO96/14408 describes an isolated DNA encoding the enzyme I-SceI. This DNA sequence can be incorporated in cloning and expression vectors, transformed cell lines and transgenic animals. The vectors are useful in gene mapping and site-directed insertion of genes.

WO00/46386 describes methods of modifying, repairing, attenuating and inactivating a gene or other chromosomal DNA in a cell through an I-SceI induced double strand break. Also disclosed are methods of treating or prophylaxis of a genetic disease in an individual in need thereof. Further disclosed are chimeric restriction endonucleases.

WO 2005/049842 describes methods and means to improve targeted DNA insertion in plants using rare-cleaving “double stranded break” inducing (DSBI) enzymes, as well as improved I-SceI encoding nucleotide sequences.

WO2006/105946 describes a method for the exact exchange in plant cells and plants of a target DNA sequence for a DNA sequence of interest through homologous recombination, whereby the selectable or screenable marker used during the homologous recombination phase for temporal selection of the gene replacement events can subsequently be removed without leaving a foot-print and without resorting to in vitro culture during the removal step, employing the therein described method for the removal of a selected DNA by microspore specific expression of a DSBI rare-cleaving endonuclease.

WO2008/037436 describe variants of the methods and means of WO2006/105946 wherein the removal step of a selected DNA fragment induced by a double stranded break inducing rare cleaving endonuclease is under control of a germline-specific promoter. Other embodiments of the method relied on non-homologous end-joining at one end of the repair DNA and homologous recombination at the other end. WO08/148,559 describes variants of the methods of WO2008/037436, i.e. methods for the exact exchange in eukaryotic cells, such as plant cells, of a target DNA sequence for a DNA sequence of interest through homologous recombination, whereby the selectable or screenable marker used during the homologous recombination phase for temporal selection of the gene replacement events can subsequently be removed without leaving a foot-print employing a method for the removal of a selected DNA flanked by two nucleotide sequences in direct repeats.

WO 2003/004659 discloses recombination systems and to a method for removing nucleic acid sequences from the chromosomal DNA of eukaryotic organisms. The invention also relates to transgenic organisms (preferably plants), containing said systems or produced by said method.

WO 2006/032426 discloses improved recombination systems and methods for eliminating maker sequences from the genome of plants. Particularly, the invention is based on use of an expression cassette comprising the parsley ubiquitin promoter, and operably linked thereto a nucleic acid sequence coding for a sequence specific DNA-endonuclease.

WO 2009/006297 discloses methods and compositions for altering the genome of a monocot plant cell, and a monocot plant, involving the use a double-stranded break inducing agent to alter a monocot plant or plant cell genomic sequence comprising a recognition sequence for the double-stranded break inducing agent.

WO 2004/006667 describes improved methods of regeneration and Agrobacterium-mediated transformation of cotton via somatic embryogenesis.

WO 2005/103271 describes methods for high efficiency plant transformation via Agrobacterium-mediated T-DNA conjugation to suspension-cultured cells or calli, employing membranes of filters as porous solid support for the co-culture of T-DNA donor and recipient.

WO 2008/112633 relates to excision of explant material comprising meristematic tissue from cotton seeds. Methods for tissue preparation, storage, transformation, and selection or identification of transformed plants are disclosed, as are transformable meristem tissues and plants produced by such methods, and apparati for tissue preparation.

Methods genome engineering using a DSBI enzyme have been applied in plant like tobacco (see e.g. Puchta et al., 1996; Townsend et al. Nature 459:442-445, 2009) and corn (see e.g. WO 2009/006297, Shukla et al. Nature 459:437-441, 2009). However, there still remains a need for the development of methods for targeted genome modification of plants that are more recalcitrant in tissue culture and transformation, such as cotton. The present invention provides a contribution over the art by providing such methods, using cotton embryogenic callus for the introduction of an endonuclease alone or in combination with a repair DNA that is to be used as a template for double stranded DNA break repair.

### SUMMARY OF THE INVENTION

In a first embodiment, the invention provides a method for modifying the genome of a cotton plant cell at a predefined site comprising the steps of
a. inducing a double stranded DNA break in the vicinity or at of said predefined site, said double stranded break being induced by the introduction into said cell of a rare-cleaving endonuclease enzyme which recognizes a recognition sequence in the vicinity of or at said predefined site;
b. selecting a plant cell wherein said double stranded DNA break has been repaired resulting in a modification in the genome at said preselected site, wherein said modification is selected from
   i. a replacement of at least one nucleotide;
   ii. a deletion of at least one nucleotide;
   iii. an insertion of at least one nucleotide; or
   iv. any combination of i.-iii.;
characterized in that said cell is comprised within embryogenic callus

In one embodiment, the endonuclease enzyme may be introduced into said cell by the delivery into said cell of a DNA molecule encoding said endonuclease enzyme.

In another embodiment, prior to step b. a foreign repair DNA molecule is delivered into said cell, said foreign repair DNA molecule being used as a template for repair of said double stranded DNA break.

In a specific embodiment, the embryogenic callus is induced from hypocotyl explants. Embryogenic callus may be induced on medium comprising active carbon. Prior to, during and after said introduction of said endonuclease enzyme, the embryogenic callus may be incubated in medium without hormones. The callus may also be incubated on solid medium prior to and after said introduction of said endonuclease enzyme. Further, during or after said introduction of the endonuclease enzyme, the embryogenic callus may be incubated for 1 to 4 days on a non-selective medium.

In one embodiment, the endonuclease enzyme encoding DNA and/or the foreign repair DNA is delivered into the cells of the embryogenic callus by particle bombardment. Bombardment may be performed with about 0.5 pmol foreign repair DNA and/or about 0.5 pmol endonuclease encoding DNA. Prior to bombardment the embryogenic callus may be incubated in a medium comprising 0.2M mannitol and 0.2M sorbitol for about 2 to about 20 hours. Further, during or after said introduction of said endonuclease enzyme, the embryogenic callus may be incubated for 1 to 4 days on a non-selective medium comprising 0.2 M mannitol.

In another embodiment, of the endonuclease enzyme encoding DNA and/or the foreign repair DNA is delivered into the cell of the embryogenic callus using Agrobacterium mediated transformation. Agrobacterium-mediated DNA transfer may be performed by co-culturing the callus with the Agrobacterium strain(s) comprising the DNA molecule(s) for about three days in a medium comprising 100 μM acetosyringone and/or 100 mg/l L-cysteine. After transformation, the calli may be incubated on a medium comprising 250 mg/L or 125 mg/L triacillin.

In a particular embodiment, the foreign repair DNA comprises at least one flanking nucleotide sequence having sufficient homology to the upstream or downstream DNA region of said predefined site to allow recombination with said upstream or downstream DNA region. The foreign repair DNA may comprise two flanking nucleotide sequences located on opposite ends of said foreign DNA, one of said flanking nucleotide sequence having sufficient homology to the upstream DNA region of said predefined site, the other flanking nucleotide sequence having sufficient homology to the downstream sequence of said predefined site to allow recombination between said flanking nucleotide sequences and said upstream and downstream DNA regions. The foreign repair DNA may also comprise a selectable marker gene and/or a plant expressible gene of interest. The plant expressible gene of interest can be selected from the group of a herbicide tolerance gene, an insect resistance gene, a disease resistance gene, an abiotic stress resistance gene, an enzyme involved in oil biosynthesis, carbohydrate biosynthesis, an enzyme involved in fiber strength or fiber length, an enzyme involved in biosynthesis of secondary metabolites.

In another embodiment, the foreign DNA consists of two flanking nucleotide sequences, one of said flanking nucleotide sequence having sufficient homology to the upstream DNA region of said predefined site, the other flanking nucleotide sequence having sufficient homology to the downstream sequence of said predefined site to allow recombination between said flanking nucleotide sequences and said upstream and downstream DNA regions.

In yet another embodiment, the preselected site is flanked by two regions with sufficient homology for recombination with each other.

The invention further provides methods for modifying the genome of a cotton plant cell at a predefined site using embryogenic callus as described above, wherein said cotton plant cell is further regenerated into a cotton plant. The thus generated cotton plant may be further crossed with another plant.

Also provided is a cotton plant cell comprising a modification at a predefined site of the genome, obtained by any of the method as described above, as well as a cotton plant or fiber or seed or propagating material of that plant, comprising a modification at a predefined site of the genome.

The invention further relates to a method of growing a cotton plant as described above, further comprising the step of applying a chemical to said plant or substrate wherein said plant is grown, as well as a method for producing a plant comprising a modification at a predefined site of the genome, comprising the step of crossing a plant consisting essentially of the plant cells as described above or a plant as described above with another plant or with itself and optionally harvesting seeds.

### FIGURE LEGENDS

FIG. 1: Schematic overview of targeted insertion/replacement through at least one-sided homologous recombination. Scissors indicate recognition sites for DSBI enzymes (I-SceI), block arrows represent promoters, arrows P3 and P4 represent primers, cross and dotted cross represent potential homologous recombination between the target construct pTCV117 and repair DNA pTIB323. After selection for hygromycin resistance (hygR) and glyphosate sensitivity (glyphS), two event groups can occur; (II) either (II) the repair DNA conferring hygromycin resistance is integrated into the genome at a random site and the EPSPS gene at the target locus is removed from bidirectional control of the 35S promoter through double stranded DNA break induction at the first (left) I-SceI site, in which case PCR amplification with primers P3×P4 does not result in a PCR product, or (I) the EPSPS gene at the target locus is truncated through homologous recombination with the 3′EPSPS fragment of the repair DNA, whereby the hygromicin resistance gene is incorporated at the target locus, and whereby at the other end various scenarios are possible depending on which of the I-SceI sites is/are used (indicated by the striped patterning), in each case allowing the region between primers P3 and P4 to be amplified.

FIG. 2: Schematic overview of targeted insertion/replacement through non-homologous end-joining. Selection for hygromycin resistance can result in either random integration of the repair DNA pTIB236 into the genome, whereby PCR amplification with primers P3×P4 and P1×P2 does not result in a PCR product, or (I-III) depending on which I-SceI site is used, insertion of the repair DNA at the site of DSBI (I and III) or replacement of the BAR gene by the repair DNA (II), resulting in PCR products with primers P3×P4 and P1×P2 of various lengths.

### DETAILED DESCRIPTION OF DIFFERENT EMBODIMENTS OF THE INVENTION

The inventors have found that targeted genome modification in cotton via double stranded DNA break induction can be efficiently performed by using embryogenic callus for the introduction of a double stranded break inducing (DSBI) enzyme, e.g. by introducing a DNA molecule encoding such and enzyme, and optionally a DNA molecule that functions as a template for repair of the double stranded DNA break, e.g. via direct DNA transfer methods (particle bombardment) or via Agrobacterium mediated DNA delivery. Using these delivery procedures into embryogenic callus, targeted insertions, replacements and deletions can be made in the nuclear genome of a cotton plant in the vicinity of the site of double stranded break induction.

As used herein, a rare-cleaving endonuclease is enzyme capable of inducing a DNA break at a particular nucleotide sequence, called the “recognition site” or “recognition sequence”, i.e. they are site specific endonucleases. They are “rare-cleaving” in the sense that due to their specific, usually long (about 12-40 nt) recognition sites they have a very low frequency of cleaving, even in the larger plant genomes, e.g. they cut less than 20×, less than 10×, less than 5× or only one time in the target genome. Rare-cleaving double stranded DNA break inducing (DSBI) enzymes are rare-cleaving endonucleases that induce a double stranded DNA break (DSB) at their recognition site. Homing endonucleases, sometimes also called meganucleases, constitute a family of such rare-cleaving endonucleases. They may be encoded by introns, independent genes or intervening sequences, and present striking structural and functional properties that distinguish them from the more classical restriction enzymes, usually from bacterial restriction-modification Type II systems. Their recognition sites have a general asymmetry which contrast to the characteristic dyad symmetry of most restriction enzyme recognition sites. Several homing endonucleases encoded by introns or inteins have been shown to promote the homing of their respective genetic elements into allelic intronless or inteinless sites. By making a site-specific double stranded break in the intronless or inteinless alleles, these nucleases create recombinogenic ends, which engage in a gene conversion process that duplicates the coding sequence and leads to the insertion of an intron or an intervening sequence at the DNA level.

A well characterized homing endonuclease is I-SceI. I-SceI is a site-specific endonuclease, responsible for intron mobility in mitochondria in Saccharomyces cerevisea. The enzyme is encoded by the optional intron Sc LSU.1 of the 21S rRNA gene and initiates a double stranded DNA break at the intron insertion site generating a 4 bp staggered cut with 3′OH overhangs. The recognition site of I-SceI endonuclease extends over an 18 bp non-symmetrical sequence (Colleaux et al. 1988 Proc. Natl. Acad. Sci. USA 85: 6022-6026). The amino acid sequence for I-SceI and a universal code equivalent of the mitochondrial I-SceI gene have been provided by e.g. WO 96/14408. WO 96/14408 further discloses a number of variants of I-SceI protein which are still functional.

PCT application PCT/EP04/013122 (incorporated herein by reference) provides synthetic nucleotide sequence variants of I-SceI which have been optimized for expression in plants. The nucleotide sequence of such synthetic I-Sce I coding regions is set forth in SEQ ID No 1 in UIPAC code. The symbols of the UIPAC code have their usual meaning i.e. N=A or C or G or T; R=A or G; Y=C or T; B=C or G or T (not A); V=A or C or G (not T); D=A or G or T (not C); H=A or C or T (not G); K=G or T; M=A or C; S=G or C; W=A or T.

A list of other rare cleaving DSBI enzymes and their respective recognition sites is provided in Table I of WO 03/004659 (pages 17 to 20) (incorporated herein by reference). These include I-Sce I, I-Chu I, I-Dmo I, I-Cre I, I-Csm I, Pl-Fli I, Pt-Mtu I, I-Ceu I, I-Sce II, I-Sce III, HO, Pl-Civ I, Pl-Ctr I, Pl-Aae I, Pl-BSU I, Pl-DhaI, Pl-Dra I, Pl-Mav I, Pl-Mch I, Pl-Mfu I, Pl-Mfl I, Pl-Mga I, Pl-Mgo I, Pl-Min I, Pl-Mka I, Pl-Mle I, Pl-Mma I, Pl-Msh I, Pl-Msm I, Pl-Mth I, Pl-Mtu I, Pl-Mxe I, Pl-Npu I, Pl-Pfu I, Pl-Rma I, Pl-Spb I, Pl-Ssp I, Pl-Fac I, Pl-Mja I, Pl-Pho I, Pl-Tag I, Pl-Thy I, Pl-Tko I or Pl-Tsp I.

Furthermore, methods are available to design custom-tailored rare-cleaving endonucleases that recognize basically any target nucleotide sequence of choice. Briefly, chimeric restriction enzymes can be prepared using hybrids between a zinc-finger domain designed to recognize a specific nucleotide sequence and the non-specific DNA-cleavage domain from a natural restriction enzyme, such as FokI. Such enzymes are generally referred to Zinc finger endonucleases (ZFEs). Such methods have been described e.g. in WO 03/080809, WO94/18313 or WO95/09233 and in Isalan et al., 2001, Nature Biotechnology 19, 656-660; Liu et al. 1997, Proc. Natl. Acad. Sci. USA 94, 5525-5530). Another way of producing custom-made rare-cleaving endonucleases or meganucleases, by selection from a library of variants, is described in WO2004/067736. Custom made meganucleases with altered sequence specificity and DNA-binding affinity may also be obtained through rational design as described in WO2007/047859. Another example of custom-designed rare-cleaving endonucleases include the so-called TALE nucleases, which are based on transcription activator-like effectors (TALEs) from the bacterial genus Xanthomonas fused to the catalytic domain of e.g. FOKI. The DNA binding specificity of these TALEs is defined by repeat-variable diresidues (RVDs) of tandem-arranged 34/35-amino acid repeat units, which can be modified to recognize specific target sequences (Christian et al., 2010, Genetics 186: 757-761, WO11/072,246, WO10/079,430 and WO11/146,121. Such custom designed rare-cleaving endonucleases are also referred to as a non-naturally occurring endonucleases.

Since the re-designed meganucleases are derived from naturally occurring endonucleases, the available potential recognition sites are not entirely random but appear to have some degree of resemblance to the nucleotide sequence originally recognized by the naturally occurring endonuclease upon which the re-designed meganuclease is based. As stated by Gao et al (2010, The Plant Journal, pp 1-11) the structure-based protein design method to modify the DNA-binding characteristics of I-CreI are based on visual inspection of the I-CreI-DNA co-crystal structure leading to a prediction of a large number of amino acid substitutions that change I-CreI base preference at particular positions in its recognition site. Individual amino acid substitutions were evaluated experimentally, and those that conferred the desired change in base preference were added to a database of mutations that can be “mixed and matched” to generate derivatives of I-CreI that recognize highly divergent DNA sites. In theory, the combinatorial diversity available using the current mutation database is sufficient to target an engineered endonuclease approximately every 1000 bp in a random DNA sequence.

Redesigned meganucleases can be based on the naturally occurring meganuclease I-CreI for use as a scaffold. I-CreI is a homing endonuclease found in the chloroplasts of Chlamydomonas rheinhardti (Thompson et al. 1992, Gene 119, 247-251). This endonuclease is a homodimer that recognizes a pseudo-palindromic 22 bp DNA site in the 23SrRNA gene and creates a double stranded DNA break that is used for the introduction of an intron. I-CreI is a member of a group of endonucleases carrying a single LAGLIDADG motif. LAGLIDADG enzymes contain one or two copies of the consensus motif. Single-motif enzymes, such as I-CreI function as homodimers, whereas double-motif enzymes are monomers with two separate domains. Accordingly, when re-designing meganucleases derived from an I-CreI scaffold to recognize a 22 bp nucleotide sequence of interest, two monomeric units are designed, each recognizing a part of the 22 bp recognition site, which are needed in concert to induce a double stranded break at the 22 bp recognition site (WO2007/047859). Concerted action may be achieved by linking the two monomeric units into one single chain meganuclease, or may also be achieved by promoting the formation of heterodimers, as described e.g. in WO2007/047859. Examples of such specifically designed meganucleases are described in e.g. EP10005926.0 and EP10005941.9 (unpublished).

Thus, for concerted action of such dimeric endonucleases, the subunits need to be dimerized in order to be able to induce a double stranded break at the preselected site in the genome. Enhanced concerted action may be achieved by linking the two monomeric units into one single chain meganuclease, or may also be achieved by promoting the formation of heterodimers, as described e.g. in WO2007/047859.

Various methods for DNA delivery into cells/tissues are known in the art, and include electroporation as illustrated in U.S. Pat. No. 5,384,253; microprojectile bombardment (biolistics) as illustrated in U.S. Pat. Nos. 5,015,580; 5,550,318; 5,538,880; 6,160,208; 6,399,861; and 6,403,865; Agrobacterium-mediated transformation as illustrated in U.S. Pat. Nos. 5,635,055; 5,824,877; 5,591,616; 5,981,840; and 6,384,301; protoplast transformation as illustrated in U.S. Pat. No. 5,508,184, electroporation, chemically-assisted transformation, liposome-mediated transformation (see, e.g., A. Deshayes, et al. (1985) EMBO J. 4:2731-7.), carbon fiber, silicon carbide fiber or aluminum borate fiber (generally termed whiskers) (see, e.g., J. Brisibe, Exp. Bot. 51 (343):187-196 (2000); Dunwell (1999) Methods Mol. Biol. 1 11:375-82; and U.S. Pat. No. 5,464,765), micro-injection (see, e.g., TJ. Reich, of al. (1986) Biotechnology 4: 1001-1004) and viral-mediated transformation (see, e.g., S. B. Gelvin, (2005) Nat. Biotechnol. 23:684-5), bombardment of plant cells with heterologous foreign DNA adhered to particles, ballistic particle acceleration, aerosol beam transformation (U.S. Patent Application No. 20010026941; U.S. Pat. No. 4,945,050; International Publication No. WO 91/00915; U.S. Patent Application No. 2002015066, WO 01/038514; all incorporated herein by reference), Lec1 transformation, PEG transformation, and various other non-particle direct-mediated methods to transfer DNA.

Targeted genome modification of cotton cells for targeted genome modification according to the invention is performed on embryogenic callus, preferable friable callus. The term “callus” or “embryogenic callus” refers to a disorganized mass of mainly embryogenic cells and cell clusters produced as a consequence of plant tissue culture. Friable callus refers to callus with a friable texture with the potential to form shoots and roots and eventually regenerate into whole plants. Such callus can further be distinguished by a parrot-green/creamy color, readily dispersed cell clumps in liquid medium, and a nodular shape. Thus, as used herein “a plant cell comprised within embryogenic callus” refers to that cell being a callus cell itself, i.e. that cell being a part of the callus tissue.

Callus can be regenerated/induced from various tissue explants, such as hypocotyl, cotyledon, immature zygotic embryos, leaves, anthers, petals, ovules, roots, and meristems, stem cells and petioles. In one embodiment of the present invention, the explant is taken from the hypocotyl or cotyledon. In one embodiment, induction of embryogenic callus is performed by incubating the explants in medium comprising active carbon for about 2 to 4 months, preferably 4 months, or at least until embryogenic callus has been formed under dim light conditions.

In one embodiment, the calli are maintained on medium without hormones during the whole procedure of callus regeneration, DNA transfer and subsequent selection and regeneration. Hormones, as used herein refers to plant hormones such as auxins e.g. 2.4-D and cytokinins (e.g. Kin). In one embodiment, cells are maintained on solid medium during the whole procedure.

In another embodiment, after DNA delivery, the calli are maintained for 1-4 days, preferably 3 days, on a non-selective medium, i.e. a medium not containing a selection compound. The non-selective medium may comprise the components of the M100 substrate. After the 1-4 days on non-selective medium, the calli may be transferred to medium that may comprise the components of the M100 substrate and a selection compound. Using a selection compound after transformation allows for the enrichment of targeted recombination events in case a repair DNA is co-delivered which comprises a selectable marker gene conferring tolerance to the selection compound. After selection of embryogenic callus, embryo induction and embryo germination may take place on a selective medium that may comprise the components of the M104 substrate and active carbon. Further embryo development may take place on a non-selective substrate that may comprise the components of the M702 substrate and plant regeneration may take place on medium comprising the components of the M700 substrate. Components of the various substrates are described below.

As used herein, DNA delivery refers to the introduction of one ore more DNA molecules into a cell. This relates to both stable transfection, wherein the introduced DNA molecule is stably integrated into the genome of the host cell as well as the transient presence of those molecule(s) in the cell. It will be clear that for performing the methods of the invention, it is not required that the cells become stably transformed with the DNA encoding the endonuclease, but transient expression of the endonuclease may already be sufficient to induce the DNA double stranded break.

Various methods for DNA delivery into cells/tissues are known in the art, and include electroporation as illustrated in U.S. Pat. No. 5,384,253; microprojectile bombardment (biolistics) as illustrated in U.S. Pat. Nos. 5,015,580; 5,550,318; 5,538,880; 6,160,208; 6,399,861; and 6,403,865; Agrobacterium-mediated transformation as illustrated in U.S. Pat. Nos. 5,635,055; 5,824,877; 5,591,616; 5,981,840; and 6,384,301; protoplast transformation as illustrated in U.S. Pat. No. 5,508,184, electroporation, chemically-assisted transformation, liposome-mediated transformation (see, e.g., A. Deshayes, et al. (1985) EMBO J. 4:2731-7.), carbon fiber, silicon carbide fiber or aluminum borate fiber (generally termed whiskers) (see, e.g., J. Brisibe, Exp. Bot. 51 (343):187-196 (2000); Dunwell (1999) Methods Mol. Biol. 1 11:375-82; and U.S. Pat. No. 5,464,765), micro-injection (see, e.g., TJ. Reich, of al. (1986) Biotechnology 4: 1001-1004) and viral-mediated transformation (see, e.g., S. B. Gelvin, (2005) Nat. Biotechnol. 23:684-5), bombardment of plant cells with heterologous foreign DNA adhered to particles, ballistic particle acceleration, aerosol beam transformation (U.S. Patent Application No. 20010026941; U.S. Pat. No. 4,945,050; International Publication No. WO 91/00915; U.S. Patent Application No. 2002015066, WO 01/038514; all incorporated herein by reference), Lec1 transformation, PEG transformation, and various other non-particle direct-mediated methods to transfer DNA.

In a specific embodiment, DNA delivery into callus comprising the cotton cells according to the invention is performed by direct DNA transfer methods, such as particle bombardment.

In one embodiment, prior to particle bombardment, calli are preplasmolysed in medium comprising mannitol and sorbitol for about 2 to about 20 hours, preferably about 2 to 4 hours.

In another specific embodiment delivery of DNA into cotton cells according to the invention is performed by Agrobacterium mediated transformation. In one embodiment, embryogenic calli are contacted with an Agrobacterium strain containing the DNA to be introduced in the cotton cells, after which the calli are co-cultivated with the Agrobacterium strain in medium comprising acetosyringone and L-cysteine for about 3 days in the dark.

In another embodiment, after the co-cultivation, transformed embryogenic calli are selected on a selection medium (i.e. comprising one or more selection compounds) further comprising triacillin.

It will be understood that the endonuclease encoding DNA and foreign repair DNA can be co-delivered to the cell or tissue (e.g. callus) sequentially (or reverse sequentially), using the same or different delivery methods, or they can be co-delivered simultaneously, e.g. whereby the foreign repair DNA and the endonuclease encoding DNA are comprised within the same mixture or even in the same molecule.

The endonuclease enzyme may but need not comprise a nuclear localization signal (NLS) (Raikhel, Plant Physiol. 100: 1627-1632 (1992) and references therein), such as the NLS of SV40 large T-antigen (Kalderon et al. Cell 39: 499-509, 1984). The nuclear localization signal may be located anywhere in the protein, but is conveniently located at the N-terminal end of the protein. The nuclear localization signal may replace one or more of the amino acids of the double stranded break inducing enzyme.

The induction of a double stranded break at a preselected site allows several potential applications. If no foreign repair DNA is introduced, the DNA region near the endonuclease recognition site may be altered by deletion, replacement or insertion of one or several to many nucleotides. In that way, the formation of small or larger deletions or insertions at the preselected site can for example inactivate the gene comprising the nucleotide sequence of the preselected site/recognition site. If the genomic DNA regions located upstream and downstream of the preselected site or recognition site have sufficient homology to each other to allow recombination between the upstream and downstream DNA region, the intervening DNA region, i.e. the DNA region between the two homologous upstream and downstream DNA region may be deleted (looped out). This can for example be used to remove previously introduced sequences such as marker genes, as e.g. described in WO 06/105946.

If the double stranded DNA break induction is accompanied by the introduction of a foreign repair DNA molecule which is used as a template, the double stranded break repair can occur basically in three ways. The repair DNA can be integrated into the genomic DNA at the DSB site by non-homologous end joining at both ends, or if one or two flanking regions with homology to the up- and/or downstream regions of the preselected site are present in the repair DNA, integration of the repair DNA can also occur (partly) through homologous recombination. As such, the double stranded break at the preselected site will also facilitate replacement of a DNA region in the vicinity of that site for a DNA region of interest e.g. as described in WO 06/105946, WO08/037,436 or WO08/148,559.

To insert a foreign DNA by homologous recombination at the preselected site, the foreign DNA may comprise at least one flanking DNA region having a nucleotide sequence which is similar to the nucleotide sequence of the DNA region upstream or downstream of the preselected site. The foreign DNA may also comprise two flanking DNA regions, located on opposite ends of the molecule and which have sufficient homology to nucleotide sequence of the DNA region upstream and downstream of the preselected site respectively to allow recombination between said flanking regions and said upstream and downstream region.

As used herein “a preselected site” or “predefined site” indicates a particular nucleotide sequence in the plant nuclear genome, located in or near the target DNA sequence at which location it is desired to insert the foreign DNA or to exchange the target DNA sequence. A person skilled in the art would be able to either choose a double stranded DNA break inducing (“DSBI”) enzyme recognizing the selected target nucleotide sequence or engineer such a DSBI endonuclease. Alternatively, a DSBI endonuclease recognition site may be introduced into the plant genome using any conventional transformation method or by conventional breeding using a plant line having a DSBI endonuclease recognition site in its genome, and any desired foreign DNA may afterwards be introduced into that previously introduced preselected target site.

As used herein “located in the vicinity” refers to the site of double DNA stranded break induction, i.e. the recognition site of the endonuclease, being located at a distance of between 500 bp, 1 kbp, 2 kbp, 3 kbp, 4 kbp, 5 kbp to 10 kbp from the predefined site, i.e. the site in the genomic DNA which is to be modified (the target site).

As used herein “a flanking DNA region” is a DNA with a nucleotide sequence having homology to the DNA regions respectively upstream and/or downstream of the target DNA sequence or preselected site. This allows to better control the precision of the intended modification. Indeed, integration by homologous recombination will allow precise joining of the foreign DNA fragment to the plant nuclear genome up to the nucleotide level.

To have sufficient homology for recombination, the flanking DNA regions of the repair DNA may vary in length, and should be at least about 10 nucleotides in length. However, the flanking region may be as long as is practically possible (e.g. up to about 100-150 kb such as complete bacterial artificial chromosomes (BACs). Preferably, the flanking region will be about 50 bp to about 2000 bp. Moreover, the regions flanking the foreign DNA of interest need not be identical to the DNA regions flanking the preselected site and may have between about 80% to about 100% sequence identity, preferably about 95% to about 100% sequence identity with the DNA regions flanking the preselected site. The longer the flanking region, the less stringent the requirement for homology. Furthermore, it is preferred that the sequence identity is as high as practically possible in the vicinity of the DSB. Furthermore, to achieve exchange of the target DNA sequence without changing the DNA sequence of the adjacent DNA sequences, the flanking DNA sequences should preferably be identical to the upstream and downstream DNA regions flanking the preselected site or the target DNA sequence destined to be exchanged. The same criteria apply for recombination between the upstream and downstream region bearing homology to each other to remove the intervening DNA sequences.

Moreover, the regions flanking the foreign DNA of interest need not have homology to the regions immediately flanking the preselected site, but may have homology to a DNA region of the genome further remote from that preselected site. Homologous recombination between the genomic DNA and the repair DNA will then result in a removal of the target DNA between the preselected insertion site and the DNA region of homology. In other words, the target DNA located between the homology regions will be substituted for the foreign DNA between the flanking regions. When the repair DNA consists of the two flanking sequences only, i.e. lacking any intervening sequences, this approach can be used to specifically delete the genomic region located between the two homology regions.

It will be clear that, in the case where homology regions are present in the foreign repair DNA, also site-specific recombinases can be used to carry out the methods of the invention. Site-specific recombinases require two recognition sites, which can be located on the same DNA molecule but also on two different DNA molecules, between which recombination occurs. Thus, a repair DNA comprising at least one such recognition site can be targeted to a genomic locus also comprising at least one such site. Examples of site-specific recombinases are well known in the art and include for instance the Cre-Lox system from bacteriophage P1 (Austin et al., 1981, Cell, 25:729-736), the Flp-Frt system from Saccheromyces, cerevisiae (Broach et al., 1982, Cell, 29:227-234), the R-RS system from Zygosaccharomyces rouxii (Araki et al., 1985, J. Mol. Biol., 182: 191-203) and the integrase from the Streptomyces phage PhiC31 (Thorpe & Smith, 1998, Proc. Natl. Acad. Sci., 95: 5505-5510; Groth et al., 2000, Proc. Natl. Acad. Sci., 97: 5995-6000).

The foreign DNA may also comprise a selectable or screenable marker, which may or may not be removed after insertion, as e.g. described in WO06/105946, WO08/037,436 and WO08/148,559.

Selectable or screenable markers” as used herein have there usual meaning in the art and include, but are not limited to plant expressible phosphinotricin acetyltransferase, neomycine phosphotransferase, glyphosate oxidase, glyphosate tolerant EPSP enzyme, nitrilase gene, mutant acetolactate synthase or acetohydroxyacid synthase gene, β-glucoronidase (GUS), R-locus genes, green fluorescent protein and the likes. Selectable markers may provide tolerance or resistance to selection compounds such as phosphinotricin, neomycin, glyphosate, hygromicin, ALS-inhibiting herbicides (e.g. sulphonyl urea and the like) or may otherwise provide means for selecting or enriching for cells wherein the desired modification has taken place, e.g. by visual means (GUS staining, fluorescence).

The selection of the plant cell or plant wherein the selectable or screenable marker and the rest of the foreign DNA molecule has been introduced by homologous recombination through the flanking DNA regions can e.g. be achieved by screening for the absence of sequences present in the transforming DNA but located outside of the flanking DNA regions. Indeed, presence of sequences from the transforming DNA outside the flanking DNA regions would indicate that the origination of the transformed plant cells is by random DNA insertion. To this end, selectable or screenable markers may be included in the transforming DNA molecule outside of the flanking DNA regions, which can then be used to identify those plant cells which do not have the selectable or screenable markers located outside of the transforming DNA and which may have arisen by homologous recombination through the flanking DNA regions. Alternatively, the transforming DNA molecule may contain selectable markers outside the flanking DNA regions that allow selection for the absence of such genes (negative selectable marker genes).

It will be clear that the methods according to the invention allow insertion of any DNA of interest including DNA comprising a nucleotide sequence with a particular nucleotide sequence signature e.g. for subsequent identification. The DNA of interest may also be one or more plant expressible gene(s) including but not limited to a herbicide tolerance gene, an insect resistance gene, a disease resistance gene, an abiotic stress resistance gene, an enzyme involved in oil biosynthesis or carbohydrate biosynthesis, an enzyme involved in fiber strength and/or length, an enzyme involved in the biosynthesis of secondary metabolites.

Herbicide-tolerance genes include a gene encoding the enzyme 5-enolpyruvylshikimate-3-phosphate synthase (EPSPS). Examples of such EPSPS genes are the AroA gene (mutant CT7) of the bacterium Salmonella typhimurium (Comai et al., 1983, Science 221, 370-371), the CP4 gene of the bacterium Agrobacterium sp. (Barry et al., 1992, Curr. Topics Plant Physiol. 7, 139-145), the genes encoding a Petunia EPSPS (Shah et al., 1986, Science 233, 478-481), a Tomato EPSPS (Gasser et al., 1988, J. Biol. Chem. 263, 4280-4289), or an Eleusine EPSPS (WO 01/66704). It can also be a mutated EPSPS as described in for example EP 0837944, WO 00/66746, WO 00/66747 or WO02/26995. Glyphosate-tolerant plants can also be obtained by expressing a gene that encodes a glyphosate oxido-reductase enzyme as described in U.S. Pat. Nos. 5,776,760 and 5,463,175. Glyphosate-tolerant plants can also be obtained by expressing a gene that encodes a glyphosate acetyl transferase enzyme as described in for example WO 02/36782, WO 03/092360, WO 05/012515 and WO 07/024,782. Glyphosate-tolerant plants can also be obtained by selecting plants containing naturally-occurring mutations of the above-mentioned genes, as described in for example WO 01/024615 or WO 03/013226. EPSPS genes that confer glyphosate tolerance are described in e.g. U.S. patent application Ser. Nos. 11/517,991, 10/739,610, 12/139,408, 12/352,532, 11/312,866, 11/315,678, 12/421,292, 11/400,598, 11/651,752, 11/681,285, 11/605,824, 12/468,205, 11/760,570, 11/762,526, 11/769,327, 11/769,255, 11/943,801 or 12/362,774. Other genes that confer glyphosate tolerance, such as decarboxylase genes, are described in e.g. U.S. patent application Ser. Nos. 11/588,811, 11/185,342, 12/364,724, 11/185,560 or 12/423,926.

Other herbicide tolereance genes may encode an enzyme detoxifying the herbicide or a mutant glutamine synthase enzyme that is resistant to inhibition, e.g. described in U.S. patent application Ser. No. 11/760,602. One such efficient detoxifying enzyme is an enzyme encoding a phosphinothricin acetyltransferase (such as the bar or pat protein from Streptomyces species). Phosphinothricin acetyltransferases are for example described in U.S. Pat. Nos. 5,561,236; 5,648,477; 5,646,024; 5,273,894; 5,637,489; 5,276,268; 5,739,082; 5,908,810 and 7,112,665.

Herbicide-tolerance genes may also confer tolerance to the herbicides inhibiting the enzyme hydroxyphenylpyruvatedioxygenase (HPPD). Hydroxyphenylpyruvatedioxygenases are enzymes that catalyze the reaction in which para-hydroxyphenylpyruvate (HPP) is transformed into homogentisate. Plants tolerant to HPPD-inhibitors can be transformed with a gene encoding a naturally-occurring resistant HPPD enzyme, or a gene encoding a mutated or chimeric HPPD enzyme as described in WO 96/38567, WO 99/24585, and WO 99/24586, WO 2009/144079, WO 2002/046387, or U.S. Pat. No. 6,768,044. Tolerance to HPPD-inhibitors can also be obtained by transforming plants with genes encoding certain enzymes enabling the formation of homogentisate despite the inhibition of the native HPPD enzyme by the HPPD-inhibitor. Such plants and genes are described in WO 99/34008 and WO 02/36787. Tolerance of plants to HPPD inhibitors can also be improved by transforming plants with a gene encoding an enzyme having prephenate deshydrogenase (PDH) activity in addition to a gene encoding an HPPD-tolerant enzyme, as described in WO 2004/024928. Further, plants can be made more tolerant to HPPD-inhibitor herbicides by adding into their genome a gene encoding an enzyme capable of metabolizing or degrading HPPD inhibitors, such as the CYP450 enzymes shown in WO 2007/103567 and WO 2008/150473.

Still further herbicide tolerance genes encode variant ALS enzymes (also known as acetohydroxyacid synthase, AHAS) as described for example in Tranel and Wright (2002, Weed Science 50:700-712), but also, in U.S. Pat. Nos. 5,605,011, 5,378,824, 5,141,870, and 5,013,659. The production of sulfonylurea-tolerant plants and imidazolinonetolerant plants is described in U.S. Pat. Nos. 5,605,011; 5,013,659; 5,141,870; 5,767,361; 5,731,180; 5,304,732; 4,761,373; 5,331,107; 5,928,937; and 5,378,824; and international publication WO 96/33270. Other imidazolinone-tolerance genes are also described in for example WO 2004/040012, WO 2004/106529, WO 2005/020673, WO 2005/093093, WO 2006/007373, WO 2006/015376, WO 2006/024351, and WO 2006/060634. Further sulfonylurea- and imidazolinone-tolerance gebnes are described in for example WO 07/024,782 and U.S. Patent Application No. 61/288,958.

Insect resistance gene may comprising a coding sequence encoding:

1) an insecticidal crystal protein from Bacillus thuringiensis or an insecticidal portion thereof, such as the insecticidal crystal proteins listed by Crickmore et al. (1998, Microbiology and Molecular Biology Reviews, 62: 807-813), updated by Crickmore et al. (2005) at the Bacillus thuringiensis toxin nomenclature, online at: http://www.lifesci.sussex.ac.uk/Home/Neil_Crickmore/Bt/), or insecticidal portions thereof, e.g., proteins of the Cry protein classes Cry1Ab, Cry1Ac, Cry1B, Cry1C, Cry1D, Cry1F, Cry2Ab, Cry3Aa, or Cry3Bb or insecticidal portions thereof (e.g. EP 1999141 and WO 2007/107302), or such proteins encoded by synthetic genes as e.g. described in and U.S. patent application Ser. No. 12/249,016; or
2) a crystal protein from Bacillus thuringiensis or a portion thereof which is insecticidal in the presence of a second other crystal protein from Bacillus thuringiensis or a portion thereof, such as the binary toxin made up of the Cry34 and Cry35 crystal proteins (Moellenbeck et al. 2001, Nat. Biotechnol. 19: 668-72; Schnepf et al. 2006, Applied Environm. Microbiol. 71, 1765-1774) or the binary toxin made up of the Cry1A or Cry1F proteins and the Cry2Aa or Cry2Ab or Cry2Ae proteins (U.S. patent application Ser. No. 12/214,022 and EP 08010791.5); or
3) a hybrid insecticidal protein comprising parts of different insecticidal crystal proteins from Bacillus thuringiensis, such as a hybrid of the proteins of 1) above or a hybrid of the proteins of 2) above, e.g., the Cry1A.105 protein produced by corn event MON89034 (WO 2007/027777); or
4) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation, such as the Cry3Bb1 protein in corn events MON863 or MON88017, or the Cry3A protein in corn event MIR604; or
5) an insecticidal secreted protein from Bacillus thuringiensis or Bacillus cereus, or an insecticidal portion thereof, such as the vegetative insecticidal (VIP) proteins listed at: http://www.lifesci.sussex.ac.uk/home/Neil_Crickmore/Bt/vip.html, e.g., proteins from the VIP3Aa protein class; or
6) a secreted protein from Bacillus thuringiensis or Bacillus cereus which is insecticidal in the presence of a second secreted protein from Bacillus thuringiensis or B. cereus, such as the binary toxin made up of the VIP1A and VIP2A proteins (WO 94/21795); or
7) a hybrid insecticidal protein comprising parts from different secreted proteins from Bacillus thuringiensis or Bacillus cereus, such as a hybrid of the proteins in 1) above or a hybrid of the proteins in 2) above; or
8) a protein of any one of 5) to 7) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation (while still encoding an insecticidal protein), such as the VIP3Aa protein in cotton event COT102; or
9) a secreted protein from Bacillus thuringiensis or Bacillus cereus which is insecticidal in the presence of a crystal protein from Bacillus thuringiensis, such as the binary toxin made up of VIP3 and Cry1A or Cry1F (U.S. Patent Appl. No. 61/126,083 and 61/195,019), or the binary toxin made up of the VIP3 protein and the Cry2Aa or Cry2Ab or Cry2Ae proteins (U.S. patent application Ser. No. 12/214,022 and EP 08010791.5);
10) a protein of 9) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation (while still encoding an insecticidal protein).

An “insect-resistant gene as used herein, further includes transgenes comprising a sequence producing upon expression a double-stranded RNA which upon ingestion by a plant insect pest inhibits the growth of this insect pest, as described e.g. in WO 2007/080126, WO 2006/129204, WO 2007/074405, WO 2007/080127 and WO 2007/035650.

Abiotic stress tolerance genes include

1) a transgene capable of reducing the expression and/or the activity of poly(ADP-ribose) polymerase (PARP) gene in the plant cells or plants as described in WO 00/04173, WO/2006/045633, EP 04077984.5, or EP 06009836.5.
2) a transgene capable of reducing the expression and/or the activity of the PARG encoding genes of the plants or plants cells, as described e.g. in WO 2004/090140.
3) a transgene coding for a plant-functional enzyme of the nicotineamide adenine dinucleotide salvage synthesis pathway including nicotinamidase, nicotinate phosphoribosyltransferase, nicotinic acid mononucleotide adenyl transferase, nicotinamide adenine dinucleotide synthetase or nicotine amide phosphorybosyltransferase as described e.g. in EP 04077624.7, WO 2006/133827, PCT/EP07/002,433, EP 1999263, or WO 2007/107326.

Enzymes involved in carbohydrate biosynthesis include those described in e.g. EP 0571427, WO 95/04826, EP 0719338, WO 96/15248, WO 96/19581, WO 96/27674, WO 97/11188, WO 97/26362, WO 97/32985, WO 97/42328, WO 97/44472, WO 97/45545, WO 98/27212, WO 98/40503, WO99/58688, WO 99/58690, WO 99/58654, WO 00/08184, WO 00/08185, WO 00/08175, WO 00/28052, WO 00/77229, WO 01/12782, WO 01/12826, WO 02/101059, WO 03/071860, WO 2004/056999, WO 2005/030942, WO 2005/030941, WO 2005/095632, WO 2005/095617, WO 2005/095619, WO 2005/095618, WO 2005/123927, WO 2006/018319, WO 2006/103107, WO 2006/108702, WO 2007/009823, WO 00/22140, WO 2006/063862, WO 2006/072603, WO 02/034923, EP 06090134.5, EP 06090228.5, EP 06090227.7, EP 07090007.1, EP 07090009.7, WO 01/14569, WO 02/79410, WO 03/33540, WO 2004/078983, WO 01/19975, WO 95/26407, WO 96/34968, WO 98/20145, WO 99/12950, WO 99/66050, WO 99/53072, U.S. Pat. No. 6,734,341, WO 00/11192, WO 98/22604, WO 98/32326, WO 01/98509, WO 01/98509, WO 2005/002359, U.S. Pat. No. 5,824,790, U.S. Pat. No. 6,013,861, WO 94/04693, WO 94/09144, WO 94/11520, WO 95/35026 or WO 97/20936 or enzymes involved in the production of polyfructose, especially of the inulin and levan-type, as disclosed in EP 0663956, WO 96/01904, WO 96/21023, WO 98/39460, and WO 99/24593, the production of alpha-1,4-glucans as disclosed in WO 95/31553, US 2002031826, U.S. Pat. No. 6,284,479, U.S. Pat. No. 5,712,107, WO 97/47806, WO 97/47807, WO 97/47808 and WO 00/14249, the production of alpha-1,6 branched alpha-1,4-glucans, as disclosed in WO 00/73422, the production of alternan, as disclosed in e.g. WO 00/47727, WO 00/73422, EP 06077301.7, U.S. Pat. No. 5,908,975 and EP 0728213, the production of hyaluronan, as for example disclosed in WO 2006/032538, WO 2007/039314, WO 2007/039315, WO 2007/039316, JP 2006304779, and WO 2005/012529.

The person skilled in the art will appreciate that, in addition to the nuclear genome, the methods of the invention may also be applied to modify e.g. the chloroplast genome or mitochondrial genome, whereby DSB induction at the predefined site and can further be enhanced by providing the correct targeting signal to the endonuclease enzyme.

It is also an object of the invention to provide cotton plant cells and plants generated according to the methods of the invention. Gametes, seeds, embryos, either zygotic or somatic, progeny or hybrids of plants comprising the genomic modification, which are produced by traditional breeding methods, are also included within the scope of the present invention. Such plants may contain a heterologous or foreign DNA sequence inserted at or instead of a target sequence or may contain a deletion, and will only be different from their progenitor plants by the presence of the particular modification.

In some embodiments, the plant cells of the invention, i.e. a plant cell comprising the T-DNA combination as well as plant cells generated according to the methods of the invention comprising the intended genomic modification, may be non-propagating cells.

The cotton plants obtained by the methods described herein may be further crossed by traditional breeding techniques with other plants to obtain progeny plants comprising the targeted modification obtained according to the present invention.

The cotton plants and seeds according to the invention may be further treated with a chemical compound, such as a chemical compound selected from the following lists:
Herbicides: Diuron, Fluometuron, MSMA, Oxyfluorfen, Prometryn, Trifluralin, Carfentrazone, Clethodim, Fluazifop-butyl, Glyphosate, Norflurazon, Pendimethalin, Pyrithiobac-sodium, Trifloxysulfuron, Tepraloxydim, Glufosinate, Flumioxazin, Thidiazuron
Insecticides: Acephate, Aldicarb, Chlorpyrifos, Cypermethrin, Deltamethrin, Abamectin, Acetamiprid, Emamectin Benzoate, Imidacloprid, Indoxacarb, Lambda-Cyhalothrin, Spinosad, Thiodicarb, Gamma-Cyhalothrin, Spiromesifen, Pyridalyl, Flonicamid Flubendiamide, Triflumuron, Rynaxypyr, Beta-Cyfluthrin, Spirotetramat, Clothianidin, Thiamethoxam, Thiacloprid, Dinetofuran, Flubendiamide, Cyazypyr, Spinosad, Spinotoram, gamma Cyhalothrin, 4-[[(6-Chlorpyridin-3-yl)methyl](2,2-difluorethyl)amino]furan-2(5H)-on, Thiodicarb, Avermectin, Flonicamid, Pyridalyl, Spiromesifen, Sulfoxaflor
Fungicides: Azoxystrobin, Bixafen, Boscalid, Carbendazim, Chlorothalonil, Copper, Cyproconazole, Difenoconazole, Dimoxystrobin, Epoxiconazole, Fenamidone, Fluazinam, Fluopyram, Fluoxastrobin, Fluxapyroxad, Iprodione, Isopyrazam, Isotianil, Mancozeb, Maneb, Metominostrobin, Penthiopyrad, Picoxystrobin, Propineb, Prothioconazole, Pyraclostrobin, Quintozene, Tebuconazole, Tetraconazole, Thiophanate-methyl, Trifloxystrobin

Cotton, as used herein refers to any existing cotton variety. For example, the cotton plant cell can be from a variety useful for growing cotton. The most commonly used cotton varieties are Gossypium barbadense, G. hirsutum, G. arboreum and G. herbaceum. Further varieties include G. africanum and G. raimondii.

Examples of cotton plants disclosed herein include those from which embryogenic callus can be derived, such as Coker 312, Coker 310, Coker 5Acala SJ-5, GSC25110, FIBERMAX 819, Siokra 1-3, T25, GSA75, Acala SJ2, Acala SJ4, Acala SJ5, Acala SJ-C1, Acala B1644, Acala B1654-26, Acala B1654-43, Acala B3991, Acala GC356, Acala GC510, Acala GAM1, Acala C1, Acala Royale, Acala Maxxa, Acala Prema, Acala B638, Acala B1810, Acala B2724, Acala B4894, Acala B5002, non Acala “picker” Siokra, “stripper” variety FC2017, Coker 315, STONEVILLE 506, STONEVILLE 825, DP50, DP61, DP90, DP77, DES119, McN235, HBX87, HBX191, HBX107, FC 3027, CHEMBRED A1, CHEMBRED A2, CHEMBRED A3, CHEMBRED A4, CHEMBRED B1, CHEMBRED B2, CHEMBRED B3, CHEMBRED C1, CHEMBRED C2, CHEMBRED C3, CHEMBRED C4, PAYMASTER 145, HS26, HS46, SICALA, PIMA S6 ORO BLANCO PIMA, FIBERMAX FM5013, FIBERMAX FM5015, FIBERMAX FM5017, FIBERMAX FM989, FIBERMAX FM832, FIBERMAX FM966, FIBERMAX FM958, FIBERMAX FM989, FIBERMAX FM958, FIBERMAX FM832, FIBERMAX FM991, FIBERMAX FM819, FIBERMAX FM800, FIBERMAX FM960, FIBERMAX FM966, FIBERMAX FM981, FIBERMAX FM5035, FIBERMAX FM5044, FIBERMAX FM5045, FIBERMAX FM5013, FIBERMAX FM5015, FIBERMAX FM5017 or FIBERMAX FM5024 and plants with genotypes derived thereof. These are suitable for applying the methods a described above.

As used herein “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a DNA region which is functionally or structurally defined may comprise additional DNA regions etc.

The term “plant” also includes progeny of plants which retain the distinguishing characteristics of the parents, such as seed obtained by selfing or crossing, e.g. hybrid seed, hybrid plants and plant parts derived therefrom.

As used herein, “plant part” includes any plant organ or plant tissue, including but not limited to fruits, seeds, embryos, fibers, meristematic regions, callus tissue, leaves, roots, shoots, flowers, gametophytes, sporophytes, pollen, and microspores.

The terms “protein” or “polypeptide” as used herein describes a group of molecules consisting of more than 30 amino acids, whereas the term “peptide” describes molecules consisting of up to 30 amino acids. Proteins and peptides may further form dimers, trimers and higher oligomers, i.e. consisting of more than one (poly)peptide molecule. Protein or peptide molecules forming such dimers, trimers etc. may be identical or non-identical. The corresponding higher order structures are, consequently, termed homo- or heterodimers, homo- or heterotrimers etc. The terms “protein” and “peptide” also refer to naturally modified proteins or peptides wherein the modification is effected e.g. by glycosylation, acetylation, phosphorylation and the like. Such modifications are well known in the art.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e. a position in an alignment where a residue is present in one sequence but not in the other, is regarded as a position with non-identical residues. The alignment of the two sequences is performed by the Needleman and Wunsch algorithm (Needleman and Wunsch 1970). The computer-assisted sequence alignment above, can be conveniently performed using standard software program such as GAP which is part of the Wisconsin Package Version 10.1 (Genetics Computer Group, Madison, Wis., USA) using the default scoring matrix with a gap creation penalty of 50 and a gap extension penalty of 3.

The sequence listing contained in the file named, “BCS11-2008_WO_ST25”, which is 59 kilobytes (size as measured in Microsoft Windows®), contains 4 sequences SEQ ID NO: 1 through SEQ ID NO: 4, is filed herewith by electronic submission and is incorporated by reference herein.

The following non-limiting Examples describe methods for modifying the genome of a cotton plant cell using a DSBI enzyme and both Agrobacterium-mediated as well as direct delivery methods on embryogenic callus.

Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR-Basics: From Background to Bench, First Edition, Springer Verlag, Germany.

All patents, patent applications and publications mentioned herein are hereby incorporated by reference, in their entireties, for all purposes.

### EXAMPLES

### Example 1

### Vector Construction

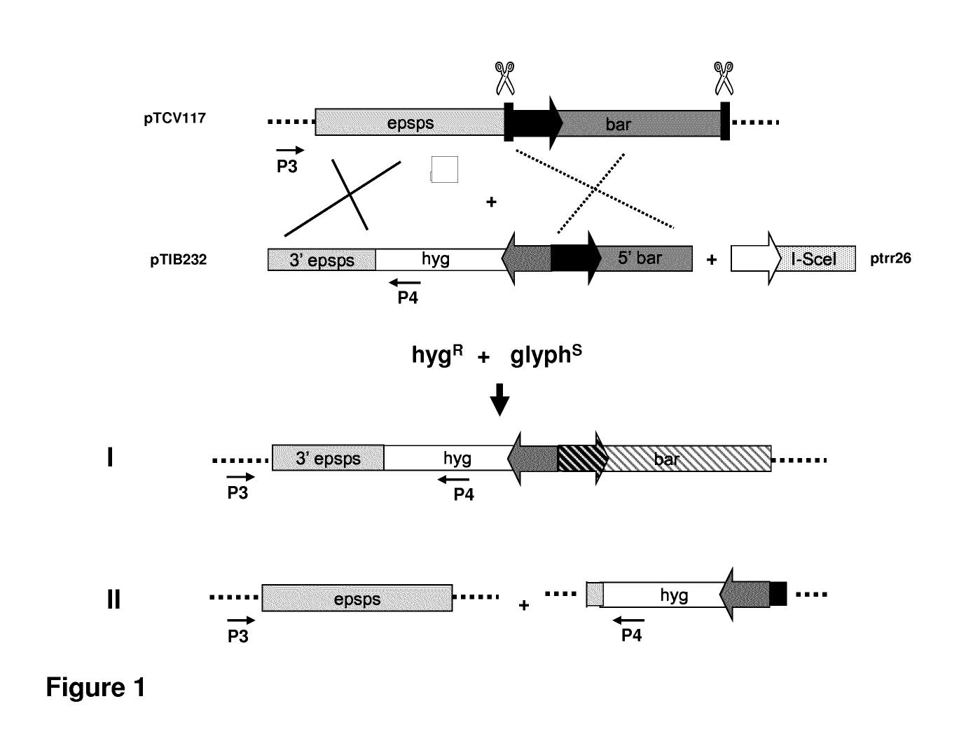

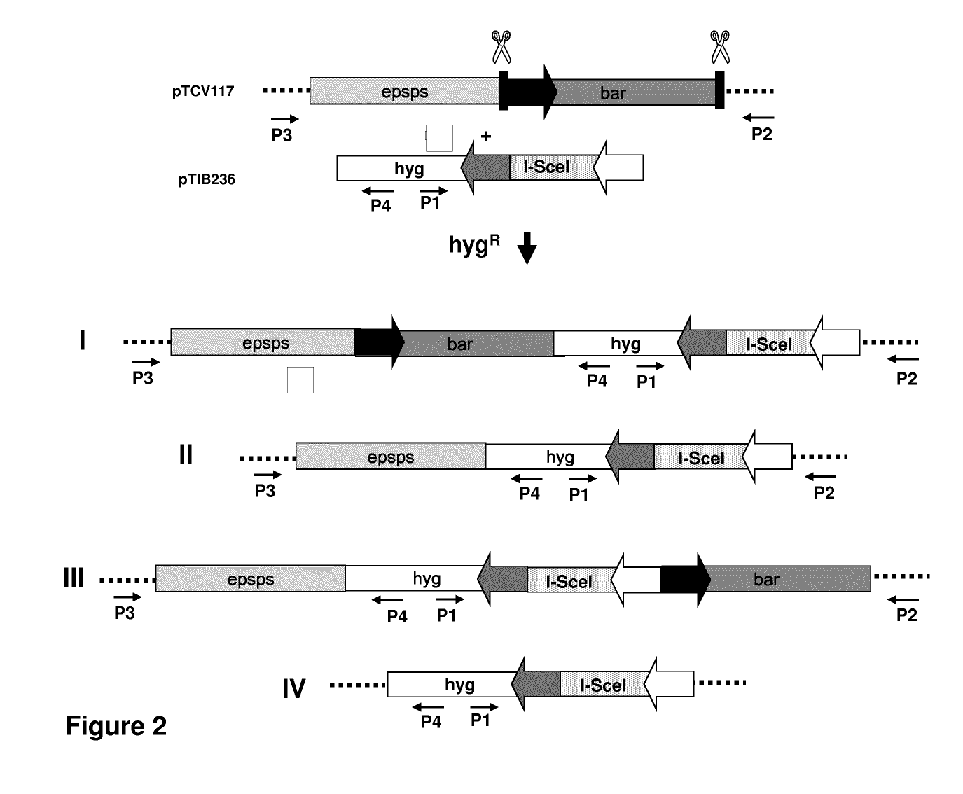

Using standard recombinant DNA techniques, the following DNA vectors were constructed comprising the following operably linked elements (schematically depicted in FIGS. 1 and 2):

Target DNA vector pTCV117 (SEQ ID NO 1):
LB: Left T-DNA border (nt 12540-12564)
I-SceI site (nt 31-14)
3′nos: sequence including the 3′ untranslated region of the nopaline synthase gene from the T-DNA of pTiT37 (Depicker et al., 1982) (nt 32-220, reverse complement)
Bar: coding region of the BAR gene of Streptomyces hygroscopicus (nt 240-791, reverse complement)
5′cab22L: sequence including the leader sequence of the chlorophyl a/b binding protein gene from Petunia hybrida (Harpster et al., 1988) (nt 801-857, reverse complement)
P35S2: 35S promoter (nt 858-1405, reverse complement)
3′ g7: 3′ end termination and polyadenylation region of gene 7 of Agrobacterium tumefaciens octopine type T-DNA (nt 1612-1409, reverse complement)
I-SceI recognition site (nt 1643-1617)
intron1 h3At: sequence including the first intron of gene II of the histone H3.III variant of Arabidopsis thaliana (Chaubet et al., 1992) (nt 1662-2127)
TPotpC: coding sequence of the optimized transit peptide, containing sequence of the RuBisCO small subunit genes of Zea mays (corn) and Helianthus annuus (sunflower), as described by Lebrun et al. (1996), (nt 2145-2510)
2mepsps: double mutant EPSPS coding region from corn (nt 2517-3852)
3′ histonAt: sequence including the 3′ untranslated region of the histone H4 gene of Arabidopsis thaliana (Chabouté et al., 1987) (nt 3871-4537)
RB: right T-DNA border (nt 4594-4618)

Repair DNA vector pTIB232 (SED ID NO 2):
LB: Left T-DNA border (nt 25-1)
bar(del1-403): 5′ fragment of the BAR gene of Streptomyces hygroscopicus (nt 46-448, reverse complement)
5′ cab22L: sequence including the leader sequence of the chlorophyl a/b binding protein gene from Petunia hybrida (Harpster et al., 1988) (nt 458-514, reverse complement)
P35S2: 35S promoter (nt 515-1062, reverse complement)
3′ g7: 3′ end termination and polyadenylation region of gene 7 of Agrobacterium tumefaciens octopine type T-DNA (nt1409-1612)
Pcsvmv XYZ: CsVMV promoter fragment (1285-1724)
5′ csvmv: leader of CsVMV promoter (nt 1725-1797)
hyg-1 Pa: Hygromycin resistance gene of E. coli (nt 1804-2829)
3′35S: 3′ 35S transcription termination and polyadenylation region (nt 2841-3065)
2mepsps(5′del): 3′ fragment of the double mutant EPSPS gene from corn (nt 3096-3501)
histonAt: 3′ end region of the histon 4 gene from Arabidopsis (nt 3520-4186)
RB: Right T-DNA border (nt 4267-4243)

Repair DNA and endonuclease expression vector pTIB236 (SEQ ID NO 3):
LB: Left T-DNA border (nt 1-25)
3′355: 35S transcription termination and polyadenylation region (nt 104-328, reverse complement)
hyg-1 Pa: hygromycine resistance gene from E. coli (nt 340-1365, reverse complement)
5′ csvmv: leader of CsVMV (nt 1372-1444, reverse complement)
Pcsvmv XYZ: CsVMV promoter fragment (1445-1884, reverse complement)
3′35S: 35S polyadenylation region (nt 1963-2097, reverse complement)
I-SceI: universal code I-SceI coding region (nt 2185-2919, reverse complement)
NLSsv40: SV40 nuclear localization signal (nt-2887-2910-, reverse complement)
5′ats1b: leader sequence from Arabidopsis thaliana rbcS ATS1A gene (nt 2936-2976, reverse complement)
P35S2: 35S promoter (nt 2976-3496, reverse complement)
RB: Right T-DNA border (nt 4592-5655)

Endonuclease expression vector ptrr26 (SEQ ID NO 4) comprising the universal code I-SceI coding region (WO 2006/074956):
RB: Right T-DNA border
P35S2: 35S promoter
5′ats1b: leader sequence from Arabidopsis thaliana rbcS ATS1A gene
NLSsv40: SV40 nuclear localization signal
I-SceI: universal code I-SceI coding region
3′35S
LB: Left T-DNA border

### Example 2

### Media and Buffers

Media and buffers used during the embryonic callus generation and transformation as described below in examples 3, 4 and 5:

Co-cultivation substrate: M100 with ½ concentration MS salts pH=5.2, +100 μM AS+100 mg/L L-cysteïne (L-cysteiïne has always to be freshly prepared and added after autoclavation)
M100 substrate: MS salts, B5 vitamins, MES 0.5 g/L, MgCl₂.6H₂O 0.94 g/L, gelrite 2 g/L, glucose 30 g/L, pH 5.8
M104 substrate: =M100 substrate+1 g/L KNO₃, pH 5.8
M700 substrate: Stewarts salts+vitamins, MgCl₂.6H₂O 0.47 g/L, gelrite 1 g/L, plant agar 2.25 g/L, sucrose 20 g/L, pH 6.8
M702 substrate: Stewarts salts+vitamins, MgCl₂.6H₂O 0.71 g/L, gelrite 1.5 g/L, plant agar 5 g/L, sucrose 5 g/L, pH 6.8
AC: active carbon 2 g/L
AS: acetosyringone 100 μM in DMSO

### Example 3

### Generation of Friable Embryogenic Callus

Cotton seeds from Coker 312 were germinated on solid germination medium M100 without hormones for 7-10 days in the dark at 28° C. Next, induction of embryogenic callus was performed by incubating hypocotyl explants from the seedlings on solid M100 medium (without hormones). After about 2 months when the wound callus at the cut surface of the hypocotyls starts to show fast proliferation, the further subculture for enrichment and maintenance of embryogenic callus is done on solid M100 medium with active carbon (2 g/L). Induction and maintenance of embryogenic callus occurs under dim light conditions (intensity: 1 to 7 μmol m⁻² sec⁻¹; photoperiod: 16H light/8H dark) at 28° C.

### Example 4

### Transformation of Cotton Embryogenic Callus by Particle Bombardment

The following procedure was followed to transform cotton embryogenic callus using particle bombardment:
Friable cotton embryogenic callus (EC) of example 3 of a target line in which to introduce a DSB induced targeted modification, is collected 2 to 3 weeks after subculture and plated in a thin layer by means of a Büchnerfilter on top of a filter paper on M100 substrate with 0.2M mannitol and 0.2M sorbitol for ˜2 to ˜20 hours prior to bombardment.
After preplasmolysis on M100 substrate with 0.2M mannitol and 0.2M sorbitol for ˜2 to ˜20 hours, the EC is bombarded with the endonuclease DNA (˜0.5 pmol) optionally in the presence of a repair DNA (˜0.5 pmol)
Bombardment conditions:
   diameter gold particles: 0.3-3 μm
   rupture disc: 1100-1350 psi
   distance to target tissue: 9 cm
   chamber vacuum ˜27 (in Hg)
   BioRAD PPS_{—}1000/He Biolistic Particle delivering system
After bombardment, the filters are transferred onto M100 substrate with 0.2 M mannitol or M100 substrate without selective agent.
After 1 to 4 days on non-selective substrate under dimlight conditions at 28° C., the filters are transferred onto selective M100 substrate with either 50 mg/L hygromycin or 1 mM glyphosate or 5 mg/L PPT
After about 2 to 3 weeks, proliferating calli are selected from the filters and further subcultured as small piles onto selective M100 substrate with either 50 mg/L hygromycin or 1 mM glyphosate or 5 mg/L PPT. After a subculture period of ˜6 weeks with ˜3 weekly subculture intervals, on selective M100 substrate under dimlight conditions at 28° C., transformed EC/somatic embryos can be selected.
A molecular screen for the identification of targeted modification events is performed at the level of transformed EC/somatic embryos.
Plant regeneration is initiated from the targeted modification events by plating EC/somatic embryos on M104 with active carbon (AC) and the corresponding selective agent under light conditions (intensity: 40 to 70 μmol m⁻² sec⁻¹; photoperiod: 16H light/8H dark) at 28° C.
After about one month individual embryos of about 0.5-1 cm are transferred on top of a filter paper on M104 with AC and the corresponding selective agent.
Further well germinating embryos are transferred onto non-selective germination substrate M702 under light conditions (intensity: 40 to 70 μmol m⁻² sec⁻¹; photoperiod: 16H light/8H dark) at 28° C.
After one to two months the further developing embryos are transferred onto M700 substrate under light conditions (intensity: 40 to 70 μmol m⁻² sec⁻¹; photoperiod: 16H light/8H dark) at 28° C. for development into small plantlets.

### Example 5

### Transformation of Cotton Embryogenic Callus by Agrobacterium-Mediated DNA Delivery

The following procedure was followed to transform cotton embryogenic callus using Agrobacterium:
Friable cotton embryogenic callus (EC) of example 3 of a target line in which to to introduce a DSB induced targeted modification is collected 2 to 3 weeks after subculture on substrate 100 and immersed for 20′ in an Agrobacterium suspension of 5×10⁸, cells/ml in M100 substrate pH 5.2, with 100 μM acetosyringone (AS). The Agrobacterium strain carries a vector containing the repair DNA and the gene encoding the endonuclease.
After 3 days co-cultivation in the dark at 24° C. on M100 with ½ concentration MS salts pH 5.2, with 100 μM AS and 100 mg/L L-cysteïne, the EC is either plated on top of a filter paper by means of a Büchner filter or transferred as small piles on M100 substrate pH5.8, 250 mg/L triacillin and a selective agent (hygromycin 50 mg/L or PPT 5 mg/L or glyphosate 1 to 1.5 mM) and incubated in dim light (intensity: 1 to 7 μmol m⁻² sec⁻¹; 16H light/8H dark) at 28° C.
After 2 to 3 weeks, calli are further subcultured as small piles onto selective M100 substrate with either 50 mg/L hygromycin or 1 mM glyphosate or 5 mg/L PPT. After a subculture period for ˜6 weeks with ˜3 weekly subculture intervals, on selective M100 substrate under dimlight conditions at 28° C., transformed EC/somatic embryos can be selected.
A molecular screen for the identification of targeted modification events is performed at the level of transformed EC/somatic embryos.
Plant regeneration is initiated from the targeted modification events by plating EC/somatic embryos on M104 with active carbon and the corresponding selective agent under light conditions (intensity: 40 to 70 μmol m⁻² sec⁻¹; photoperiod 16H light/8H dark) at 28° C. From this step on, the substrates do not contain anymore antibiotics.
After about one month individual embryos of about 0.5-1 cm are transferred on top of a filter paper on M104 with active carbon (AC) and the corresponding selective substrate.
Further germinating embryos are transferred on non-selective germination substrate M702 under light conditions (intensity: 40 to 70 μmol m⁻² sec⁻¹; photoperiod (16H light/8H dark at 28° C.
After one to two months the further developing embryos are transferred onto M700 substrate under light conditions (intensity: 40 to 70 μmol m⁻² sec⁻¹; photoperiod (16H light/8H dark) at 28° C. for development into small plantlets.

### Example 6

### Generation of Target Plants for the Evaluation of Targeted Genome Engineering

Transgenic cotton plants comprising the pTCV117 target DNA vector were generated by Agrobacterium as described in example 5. The pTCV117 vector comprises a functional 35S-driven bar gene located between two I-SceI recognition sites and a promoterless EPSPS gene (see FIG. 1). To evaluate targeted recombination, it was originally intended to transform these plants with a 35S-I-SceI expression cassette and a repair DNA with homology regions to the target DNA for restoration of the promoter-less epsps gene by insertion of a histon promoter, thereby resulting in the acquisition of glyphosate tolerance. However, these pTCV117 plants appeared to already have high levels of tolerance to glyphosate, probably due to bidirectional transcriptional activity of the 35S promoter, thereby making the assay unusable.

### Example 7

### Targeted Insertion or Replacement Through Homologous Recombination

Thus, a new repair DNA pTIB232 with homology regions to the target DNA was constructed, comprising a functional CSVMV-driven hygromycin resistance gene flanked at one end by a 3′ epsps gene fragment (allowing homologous recombination with the EPSPS gene at the target locus, thereby replacing the 5′ part of the EPSPS gene by the hygromycin gene) and at the other end flanked by a 35S-promoter linked to a 5′ bar gene fragment), as schematically indicated in FIG. 1. pTCV117 target plants were transformed with the pTIB232 vector and the ptrr26 vector comprising a universal code I-SceI coding region operably linked to a 35S promoter using particle bombardment as described in example 4.

First, transformants were screened for hygromycin tolerance, as this indicates insertion of the repair DNA pTIB232. HygR events were subsequently evaluated for loss of glyphosate resistance, which is indicative of recombination at the target locus. The 36 thus obtained hygR and GlyS recombinants were further characterized by PCR analysis using primer pair P3×P4 recognizing the genomic region upstream of the EPSPS gene and the hygromycin gene (FIG. 1). This resulted in the identification of 8 potential correct gene targeting events (replacement of the 5′ part of the epsps gene by the hygromycin gene by at least one-sided homologous recombination: configuration I in FIG. 1), which were subsequently confirmed to be indeed correct gene targeting events by sequence analysis of the PCR product obtained by the primer set P3, P4).

### Example 8

### Targeted Insertion or Replacement Through Non-Homologous End-Joining

pTCV117 plants were transformed with repair DNA and endonuclease expression vector pTIB236, comprising a functional hygR gene and a functional universal code I-SceI encoding gene, with no homology to the target site (see FIG. 2), using Agrobacteruim-mediated DNA transfer as described in example 5.

The 200 hygR events that were thus obtained, were analyzed by PCR using 2 primer pairs P1×P2 and P3×P4; the primer pair P1×P2 recognizing the hygR gene and the genomic region downstream of the bar gene and the other pair recognizing the genomic region upstream of the EPSPS gene and the hygromycin gene (FIG. 2). This resulted in the identification of 17 potential targeted insertion/replacement events. Sequence analysis done on 4 of these events showed that they were indeed targeted insertion/replacement events (configuration I, II in FIG. 2).

### Example 9

### Targeted Insertion or Replacement Via Non-Homologous End Joining Using Agrobacterium-Mediated Transformation

Target line G4 GH198-01901 was transformed as described in Example 5 with Agrobacterium strain A5280=EHA101(pTIB236) comprising the repair DNA and endonuclease expression vector pTIB236.

Hygromycin resistant (HygR) events were selected of which 200 were analyzed by PCR for insertion of the hyg gene at the target I-SceI site(s) using primer pairs P1×P2 and P3×P4. Of these 200 events, about 10% were found to be targeted events. Sequence analysis of some of these targeted events revealed that either the bar gene had been replaced by the hyg gene or that the hyg gene had inserted next to the bar gene (stacked event).

### Example 10

### Targeted Double-Stranded Break Induction Using a Custom Designed Meganuclease

Embryogenic calli from PPT-resistant cotton plants containing a chimeric gene comprising the bar gene under control of the CSVMV promoter were transformed via particle bombardment with a vector comprising a chimeric gene encoding a custom engineered meganuclease recognizing a target sequence in the bar gene, either as a single chain (pCV170: SEQ ID NO. 5) or as a heterodimer (pCV177: SEQ ID NO. 6). A co-delivery was done of the meganuclease vector with a vector containing the 2mEPSPS gene under control of a plant-expressible promoter conferring glyphosate tolerance as a selectable marker gene. About 3000 glyphosate resistant calli were obtained of which 85 events appeared PPT sensitive, indicating a disruption of the bar gene. Of these, 79 events were characterized for genotype by PCR using primers flanking the target site and subsequent sequencing of the PCR product (table 1).

TABLE 1
Characterization of PPT-sensitive glyphosate-resistant
transformation events. The absence of a PCR product is
indicative of a large deletion around the target site.

| | | | |
|---|---|---|---|
| PCR product obtainable | # events | change at target site | # events |

pCV170 (sc)

| | | | |
|---|---|---|---|
| no | 11 | large deletion | 11 |
| yes | 8 | no mutation | 6 |
| | | replacement/insertion | 1 |
| | | deletion | 1 |

pCV177 (hd)

| | | | |
|---|---|---|---|
| no | 33 | large deletion | 33 |
| yes | 27 | no mutation | 19 |
| | | insertion | 4 |
| | | deletion | 4 |

Thus, these results demonstrate that it is possible to induce a targeted double stranded DNA break at the desired position with both a single chain as well as a heterodimeric custom-designed meganuclease and that targeted deletion, replacement and insertion events can be obtained using such meganucleases in cotton.

1. A method for modifying the genome of a cotton plant cell at a predefined site comprising the steps of
a. inducing a double stranded DNA break in the vicinity of or at said predefined site, said double stranded break being induced by the introduction into said cell of a rare-cleaving endonuclease enzyme which recognizes a recognition sequence in the vicinity of or at said predefined site;
b. selecting a plant cell wherein said double stranded DNA break has been repaired resulting in a modification in the genome at said preselected site, wherein said modification is selected from
i. a replacement of at least one nucleotide;
ii. a deletion of at least one nucleotide;
iii. an insertion of at least one nucleotide; or
iv. any combination of i.-iii.;
characterized in that said cell is comprised within embryogenic callus.

2. The method of claim 1, wherein said endonuclease enzyme is introduced into said cell by the delivery into said cell of a DNA molecule encoding said endonuclease enzyme.

3. The method of claim 1, wherein prior to step b. a foreign repair DNA molecule is delivered into said cell, said foreign repair DNA molecule being used as a template for repair of said double stranded DNA break.

4. (canceled)

5. The method of claim 1, wherein said embryogenic callus is induced on medium comprising active carbon.

6. The method of claim 1, wherein said embryogenic callus is incubated in medium without hormones prior to, during and after said introduction of said endonuclease enzyme.

7. (canceled)

8. The method of claim 1, wherein said embryogenic callus is incubated for 1 to 4 days on a non-selective medium during or after said introduction of said endonuclease enzyme.

9. The method of claim 2 or 3, wherein said DNA delivery is performed by particle bombardment.

10-12. (canceled)

13. The method of claim 2 or 3, wherein said DNA delivery is performed using Agrobacterium.

14-15. (canceled)

16. The method according to claim 3, wherein said foreign repair DNA comprises at least one flanking nucleotide sequence having sufficient homology to the upstream or downstream DNA region of said predefined site to allow recombination with said upstream or downstream DNA region.

17. The method according to claim 3, wherein said foreign repair DNA comprises two flanking nucleotide sequences located on opposite ends of said foreign DNA, one of said flanking nucleotide sequence having sufficient homology to the upstream DNA region of said predefined site, the other flanking nucleotide sequence having sufficient homology to the downstream sequence of said predefined site to allow recombination between said flanking nucleotide sequences and said upstream and downstream DNA regions.

18. The method according claim 3, wherein said foreign repair DNA comprises a selectable marker gene.

19. The method according to claim 3, wherein said foreign DNA comprises a plant expressible gene of interest, said plant expressible gene of interest optionally being selected from the group of a herbicide tolerance gene, an insect resistance gene, a disease resistance gene, an abiotic stress resistance gene, an enzyme involved in oil biosynthesis, carbohydrate biosynthesis, an enzyme involved in fiber strength or fiber length, an enzyme involved in biosynthesis of secondary metabolites.

20. (canceled)

21. The method according to claim 3, wherein said foreign DNA consists of two flanking nucleotide sequences, one of said flanking nucleotide sequence having sufficient homology to the upstream DNA region of said predefined site, the other flanking nucleotide sequence having sufficient homology to the downstream sequence of said predefined site to allow recombination between said flanking nucleotide sequences and said upstream and downstream DNA regions.

22. The method of claim 3, wherein said preselected site is flanked by two regions with sufficient homology for recombination with each other.

23. The method according to claim 1, wherein said cotton plant cell is further regenerated into a cotton plant.

24. The method according to claim 23, wherein said plant is further crossed with another plant.

25. A cotton plant cell comprising a modification at a predefined site of the genome, obtained by the method according to claim 1.

26. A cotton plant or fiber or seed or propagating material thereof consisting essentially of the cotton plant cells of claim 25.

27. A method of growing a cotton plant according to claim 26, comprising the step of applying a chemical to said plant or substrate wherein said plant is grown.

28. A method for producing a plant comprising a modification at a predefined site of the genome, comprising the step of crossing a plant according to claim 26 with another plant or with itself and optionally harvesting seeds.
